(19) United States
(12) Patent Application Publication
(10) Pub. No.: US 2013/0084334 A1
(43) Pub. Date: Apr. 4, 2013

(54) PHARMACEUTICAL COMPOSITIONS HIGHLY DOSED WITH BIOTIN

(71) Applicants: Frédéric Sedel, Paris (FR); Agnès Bellanger, Paris (FR)

(72) Inventors: Frédéric Sedel, Paris (FR); Agnès Bellanger, Paris (FR)

(73) Assignee: Assistance Publique - Hopitaux de Paris, Paris (FR)

(21) Appl. No.: 13/639,752

(22) PCT Filed: Apr. 5, 2011
(86) PCT No.: PCT/EP2011/055273

§ 371 Date: Nov. 14, 2012

(30) Foreign Application Priority Data
Apr. 6, 2010 (FR) 10/52589

(51) Int. Cl. A61K 31/407

(52) U.S. Cl. CPC A61K 31/407
USPC 424/474; 548/303.7; 514/393

(57) ABSTRACT

The invention relates to novel pharmaceutical compositions highly dosed with biotin, as well as to the use thereof in treating visual impairments which are related, in particular, to optic atrophy.

The invention relates to novel pharmaceutical compositions containing a high dose of biotin, and also to the use thereof in treating visual impairments, which are related in particular to optic atrophy.

Biotin (or vitamin H) is a ubiquitous water-soluble vitamin which is naturally found in many foods, including offal, eggs and certain vegetables. Biotin taken in via food comes from biocytin (biotin-lysine dimer) which is a product of the degradation of proteins by digestive proteases and peptidases. Free biotin is produced in the intestinal lumen through the action of biotinidase. The biotin is then transported by several transporters, in particular the sodium-dependent multivitamin transporter (SMVT). Biotin is eliminated essentially in the urine, in the form of catabolites. Acquired deficiencies can be observed in malnourished individuals, or in the event of poisoning with avidin contained in egg white (avidin binds to biotin in the lumen of the digestive tract and prevents its absorbtion).

In mammals, biotin acts as a cofactor for four metabolism carboxylases involved in several key steps of energy metabolism, including pyruvate carboxylase (neoglucogenesis), 3-methylcrotonyl CoA and propionyl CoA carboxylases (catabolism of certain amino acids which provide the Krebs cycle with intermediate metabolites); and acetyl CoA carboxylase (fatty acid synthesis).

Over the past few years, it has also been shown that biotin can regulate the expression of many genes via a mechanism of biotinylation/debiotinylation of histones, which are protein structures that regulate DNA conformation and, in so doing, the access of certain regions of the genome to transcription factors. It appears that a large number of genes in which the expression is regulated by biotin encode proteins involved in energy metabolism (Zempleni et al., 2009).

In humans, two hereditary metabolic diseases directly affect biotin metabolism: biotinidase deficiency and holocarboxylase synthetase deficiency (Zempleni et al., 2009). Biotinidase is essential for the release of free biotin from the biocytin generated by proteolysis. Holocarboxylase synthetase is, itself, involved in the binding of free biotin to 4 biotin-dependent apocarboxylases (3-methylcrotonyl CoA carboxylase, propionyl CoA carboxylase, pyruvate carboxylase and acetyl CoA carboxylase). These two diseases have polymorphic clinical presentations, but the first signs generally appear in infancy: metabolic acidosis, epileptic fits, hypotonia, consciousness disorders, cerebellar ataxia, perceptive deafness, skin rash, alopecia. These symptoms can be improved spectacularly (in particular in the case of biotinidase deficiency) with small doses of biotin (between 5 and 20 mg/day). Late forms of biotinidase deficiency beginning at adolescence or in adulthood have also been reported, in particular in some patients with medullary involvement or optic atrophy (Ramaekers et al., 1993; Wolf et al., 1998). In certain cases, these neurological problems were reversible with biotin.

Moreover, a genetic disease, of autosomal recessive transmission, not directly related to biotin metabolism, and called “biotin-responsive basal ganglia disease” (BBGD, OMIM # 607483) was described for the first time in 1998 in 10 patients originating from the Middle East (Ozand et al., 1998). The patients suffering from BBGD exhibited subacute episodes of encephalopathy which are often triggered by febrile episodes and were characterized by confusion, epilepsy, external ophthalmoplegia, dysphagia, and generalized weakness sometimes leading to coma or even to death. The administration of very high doses of biotin (10 mg/kilo/day) during these attacks enabled partial or complete recovery in a few days. In the absence of treatment, the patients exhibited serious neurological after-effects, in particular permanent widespread dystonia in relation to bilateral damage of the central gray nuclei. The gene responsible for this syndrome has recently been identified: it is SLC19A3 encoding the hTHTR2 protein which is a secondary thiamine transporter (Zeng et al., 2005). Since biotin is not a substrate for hTHTR2 (Subramanian et al., 2006), the precise mechanism by which biotin improves the clinical phenotype remains a mystery.

One hypothesis could be that biotin at very high doses activates the expression of the SLC19A3 gene, thus making it possible to restore the function of the defective transporter. This “transcriptional activation therapy” hypothesis is based on the following observations:
(1) biotin regulates many genes (Zempleni, 2009), and
(2) the expression of the SLC19A3 gene decreases in the event of biotin deficiency (Vlasova et al., 2005), indicating that the activity of this gene is regulated (directly or indirectly) by biotin.

Another hypothesis would be that the forced activation of biotin-dependent enzymes, by high doses of biotin, results in activation of the Krebs cycle via the provision of anaplerotic substrates generated by these enzymes, which could compensate for the thiamine transport deficit caused by the mutations of SLC19A3.

Biotin is present as an active ingredient in a certain number of medicaments. However, these compounds contain a low amount of biotin per dose.

In most cases, these medicaments also contain other active ingredients (in particular other vitamins). These products can be used orally. These products which have several active ingredients contain less than 1 mg of biotin (between 0.15 and 0.2 mg) per unit dose (lozenge or tablet).

A medicament sold in France (Bayer® Biotine) is in the form of tablets or an injectable solution and contains only biotin, as active ingredient, and also excipients. This medicament is used as an adjuvant treatment for diffuse alopecia. Bayer biotin contains 5 mg of biotin per unit dose (tablet or injectable vial).

In the context of the present invention, a novel pharmaceutical form (novel dosage) of biotin has been developed, which allows oral administration of a large amount of biotin for each unit dose. These medicaments can be used for treating diseases which cause visual impairments through optic neuropathy, and in particular certain forms of leukoencephalopathy exhibiting original clinical signs.

The high doses of biotin administered by the compositions according to the invention allow an improvement in the clinical signs, the hypothesis for this improvement being that it is linked to good assimilation of the biotin provided. It should be noted that no toxic effect of the biotin at very high doses has been described in the medical literature or in the patients treated by the inventors.

In a first aspect, the invention relates to a composition for oral administration, containing at least 20 mg, preferably at least 40 mg, of biotin. This composition is preferentially for pharmaceutical use, and is therefore a medicament. It is understood that each unit dose of the composition according to the invention contains at least 20 mg, preferably at least 40 mg of biotin, as active ingredient.

In one preferred embodiment, said composition contains at least 50 mg of biotin. In another embodiment, said composition contains at least 75 mg of biotin. In the preferred embodiment, said composition contains at least 100 mg of biotin.

In one particular embodiment, the composition according to the invention contains biotin as sole active ingredient, and also excipients, without any other active ingredient.

An excipient should be understood to mean any compound forming part of the formulation which is intended to act as a simple support, i.e. which is not intended to have a biological effect.

The composition according to the invention can be in any form known in the art. In particular, it is in the form of gel capsules, tablets (optionally film-coated), pills or lozenges. In another embodiment, it is in the form of a syrup. Said syrup contains an amount of biotin such that it contains at least 20 mg, preferably at least 40 mg of biotin per unit dose. The biotin concentration in this syrup depends on the unit dose that it is desired to give to the patient.

Excipients that can be used by those skilled in the art are well known in the art. Talc (E553b), microcrystalline cellulose, lactose, starch (in particular corn starch), magnesium stearate (E572) and stearic acid (E570) can thus be chosen.

When the composition according to the invention is prepared in the form of gel capsules, a preferred excipient is microcrystalline cellulose.

When the composition is in the form of a film-coated tablet, said film coating can be formed from any substance known in the art, such as hypromellose (E464), ethylcellulose, macrogol, talc (E553b), titanium dioxide (E171) or iron oxide (E172).

The active ingredient may also be colored (with any acceptable coloring, such as cochineal), which makes it possible to verify that the biotin is well dispersed in the excipient.

In another aspect, the invention relates to an injectable composition, containing at least 20 mg, preferably at least 40 mg of biotin per unit dose, preferably at least 50 mg, more preferably at least 75 mg, most preferably at least 100 mg of biotin per unit dose.

This injectable composition can be in the form of a vial containing the biotin, and also acceptable excipients. The biotin concentration is adjusted according to the vial volume envisioned. Certain excipients which improve the solubility of biotin can be used.

The excipients that can be used for producing injectable compositions are well known in the art. Mention may particularly be made of sodium dihydrogen phosphate, sodium bicarbonate (E550i), methyl para-hydroxybenzoate (E218) and propyl para-hydroxybenzoate (E216), which can be used together in proportions that those skilled in the art are capable of determining. The water used is water for injection. The injection is preferably carried out intramuscularly. It can also be carried out intravenously.

The compositions containing a high dose of biotin are particularly advantageous and suitable for use in the treatment of a visual impairment or of a visual atrophy. This treatment may be a main treatment or an adjuvant treatment for a main treatment, aimed at attacking the causes of the visual atrophy. The invention also relates to the use of biotin for preparing a medicament intended for treating a visual impairment or atrophy (or any other pathological condition mentioned below), and also to the methods for treating these pathological conditions by administering biotin.

The compositions containing a high dose of biotin are particularly advantageous and suitable for stabilizing the clinical condition of a patient suffering from a visual impairment or atrophy.

For such a use, it is preferable for the amount of biotin administered to the patient to be at least equal to 1 mg/kg/day, preferably 3 mg/kg/day, more preferably 5 mg/kg/day, or at least equal to 7.5 mg/kg/day, or even around 10 mg/kg/day. Between 100 and 700 mg of biotin per day are administered to patients, generally between 200 and 500 mg per day, generally around 300 mg per day.

Visual atrophy (or optic atrophy) is generally due to atrophy of the optic nerve, accompanied by a modification of the visual field and a decrease in visual acuity. It can be caused by an inflammatory, tumor, vascular or toxic process.

In one particular embodiment, the visual atrophy is observed in the absence of a clear etiology as mentioned above.

In this embodiment, the visual atrophy is a symptom linked to a particular leukoencephalopathy, i.e. involvement of the white matter of the brain.

This leukoencephalopathy can be characterized by the following elements: involvement of the following regions of the white matter of the brain: periventricular white matter, optic radiations, corticospinal tracts, cerebellar peduncles, as can be observed by MRI of the brain, and an elevation in the choline peak in the centrum semiovale, as can be observed by nuclear magnetic resonance spectroscopy (NMRS).

Thus, specific MRI abnormalities are observed which are not found during other leukoencephalopathies of metabolic origin (reviewed in Sedel at al., 2008): (1) corticospinal tract hypersignal; (2) optic radiation hypersignal; (3) cerebellar peduncle hypersignal; (4) moderate hypersignal of the periventricular white matter.

From the electrophysiological point of view, the patients exhibit abnormalities evoking unilateral or bilateral involvement of the optic nerves, even in the absence of a decrease in visual acuity.

An inflammatory reaction (hypercellularity >4 elements/mm³) is also often observed in patients.

This leukoencephalopathy sensitive to high doses of biotin is thus accompanied by unilateral or bilateral involvement of the optic nerves, which may be symptomatic (decrease in visual acuity) or subclinical (detected only on the visual evoked potentials, without any clinical sign), which improves with treatment. The cerebellar syndrome and the elevation of the choline peak observed by NMRS improve with treatment.

The diagnosis of this “biotin-sensitive leukoencephalopathy” is based on clinical, radiological and neurophysiological criteria:
a) Clinical criteria: disease progressing via attacks which take hold over the course of a few days and during which the patient can exhibit the following symptoms: cerebellar syndrome, unilateral decrease in visual acuity. Between these subacute episodes, the patients can exhibit headaches, psychiatric problems, and after-effects of the prior attacks: permanent cerebellar syndrome, permanent unilateral or bilateral decrease in visual acuity.
b) Radiological criteria: the brain MRI shows a characteristic leukoencephalopathy which involves the following regions of the white matter of the brain: periventricular white matter, optic radiations, corticospinal tracts, cerebellar peduncles. The MRI of the optic nerves can show unilateral or bilateral optic atrophy. The nuclear magnetic resonance spectroscopy (NMRS) shows an elevation of the choline peak in the centrum semiovale.
c) Neurophysiological criteria: the study of the visual evoked potentials can show a bilateral increase in the P100 waves, related to involvement of both optic nerves, or an absence of a P100 wave in severe cases. The visual evoked potential (VEP) is the electrical response of the cortex which is caused by a visual stimulation. The VEPs result from the recording of the variations in potentials generated by the bioionic activity of the occipital cortex subsequent to a visual stimulus of which a parameter varies over time. The VEPs study the macular and perimacular function and also the conduction of the visual pathways.

The composition according to the invention is administered to patients exhibiting the criteria defined above, at a dose as mentioned above, in particular 100 mg three times a day for three months.

In the event of a definite improvement in one of the parameters after clinical, radiological (MRI+NMRS) and electrophysiological reevaluation, the treatment can be continued. The biotin dose can be adjusted (increased or decreased).

If none of the parameters has definitely improved, but there is a strong diagnostic suspicion (clinical, MRI, strongly suggestive visually evoked potentials), the therapeutic treatment is continued for a further three months (with the biotin dose optionally being adjusted), at the end of which a further clinical, radiological (MRI+NMRS) and electrophysiological evaluation is carried out.

The compositions according to the invention can also be used for treating patients suffering from other pathological conditions:
a) Suspicion of BBGD: episodes of encephalopathy triggered by febrile episodes and during which the brain MRI shows lesions in FLAIR/T2 hypersignal of the central gray nuclei (putamen and caudate nuclei). A dosage of 10 mg/kilo/day should be proposed during the episodes of encephalopathy, in combination with vitamin B1 (500 mg/day) (Debs et al., 2010). This therapeutic test should be accompanied by a genetic study with a search for mutations in the SLC19A3 gene.
b) Suspicion of biotin-sensitive pathological condition: neurological affection “without diagnosis” progressing via subacute episodes and combining diversely the following clinical signs: unilateral or bilateral optic atrophy, subacute cerebellar syndrome, involvement of the central gray nuclei.
c) Other neurological pathological condition potentially linked to an energy metabolism disorder: mention may be made of Alzheimer's disease,

Huntington's chorea, Parkinson's disease, or certain symptomatic epilepsies. The high-dose biotin can thus be used as an adjuvant treatment for reducing the symptoms observed for these diseases.

### DESCRIPTION OF THE FIGURES

FIG. 1: MRI and NMR spectroscopy controls carried out on patient 1 before (FIG. 1A) and after treatment (FIG. 1B). On the MRI carried out before treatment, a leukoencephalopathy affecting the cerebellar peduncles (PC), the corticospinal tracts (CS), the optic radiations (RO) and the periventricular white matter (PV) is observed. The NMR spectroscopy shows an increase in the choline peak (Cho, peak furthest to the left), which should be at a height similar to the peak located just to its right. After treatment, the signs of leukoencephalopathy and the choline peak have clearly decreased.

FIG. 2: MRI and NMR spectroscopy controls carried out on patient 2 before (FIG. 2A) and after treatment (FIG. 2B). On the MRI carried out before treatment, a leukoencephalopathy affecting the cerebellar peduncles (PC), the corticospinal tracts (CS), the optic radiations (RO) and the periventricular white matter (PV) is observed. The NMR spectroscopy shows an increase in the choline peak (Cho). After treatment, the choline peak has decreased.

FIG. 3: MRI controls carried out on patient 3 before (FIG. 3A) and after treatment (FIG. 3B). On the MRI carried out before treatment, a leukoencephalopathy affecting the cerebellar peduncles (PC), the corticospinal tracts (CS), the optic radiations (RO) and more weakly the periventricular white matter (PV) is observed. After treatment, the signs of leukoencephalopathy have disappeared.

The following examples show the advantage of a treatment with the preparations according to the invention.

### EXAMPLES

### Preliminary Example: Preparation of Gel Capsules Containing a High Dose of Biotin

The biotin, the raw material, is obtained from a pharmaceutical wholesale company, LA COOPER (Cooperation Pharmaceutique Francaise) [French Pharmaceutical Cooperation] in Melun. Before mixing with the excipient (microcrystalline cellulose), a pinch of cochineal is added to the active ingredient (tracer for good distribution in the mixture). The mixture is then distributed into No. 1 gel capsules (0.50 ml).

For 100 gel capsules containing a dose of 100 mg, the following mixture is prepared:
biotin: 10 g
cochineal: a pinch
microcrystalline cellulose: qs to 50 ml.

### Example 1

A female patient (54 years old in 2010) presented a decrease in visual acuity of the left eye, followed by a decrease in visual acuity of the right eye 10 days later (May 2002). The decrease in visual acuity is painless, but was preceded by neck pain and headaches.

Optical atrophy took hold rapidly. In December 2002, she exhibited, over the course of several days, balance problems, sphincter problems (dysuria) and paresthesia of the four limbs.

The neurological examination demonstrated sharp reflexes in the four limbs with a bilateral Babinski sign, and also a left lower limb kinetic cerebellar syndrome.

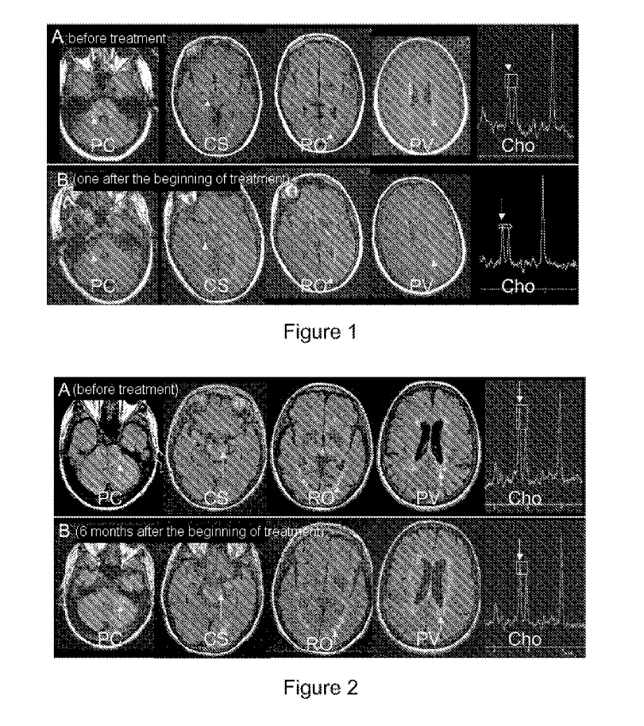

The MRI carried out at this time shows a leukoencephalopathy affecting the periventricular white matter, the corticospinal tracts at the level of the internal capsules and of the brain stem with a clear hypersignal, the optic radiations and cerebellar peduncles, especially on the right. The NMR spectro on Dec. 6, 2002, does not show any elevation of the choline peak. The medullary MRI is normal. Several MRI controls in July 2003 and May 2004 show a superposable appearance (FIG. 1A). However, the choline peak is abnormally elevated on the NMR spectro of May 2004.

In July 2003, she can count fingers at 20 cm on the right and see the hand move on the left.

In May 2004, the visual acuity numbers are 1.6/10 on the right and 1/50^{th} on the left. This visual acuity remains stable until April 2006, at which time a treatment with biotin at the dose of 20 mg/day is introduced.

After two months of treatment, the patient has the impression of seeing a little better. After three months of treatment (July 2006), the visual acuity numbers are 4/10 on the right and still 1/50 on the left. This initial improvement is maintained at the 7-month control (November 2006).

In December 2006, the treatment is increased to 100 mg/day.

After two weeks, the patient begins to be able to read the headlines of a newspaper.

In July 2007 (after 15 months of treatment), the visual acuity is scored at 6/10 on the right and at 1/20 on the left. The visual VEPs then show a delayed right-side cortical response at +4.5DS (the response being zero before treatment).

The patient also indicates an improvement in her balance problems. The July (2007) MRI shows a normalization of the choline peak, and also a clear decrease in the leukoencephalopathy signal intensity (FIG. 1B).

### Example 2

A treatment based on high-dose biotin was given to a 72-year-old patient (in 2010). This patient had complained of migraines since the age of 20.

In 2004 (66 years old), she exhibited psychiatric problems of persecution delirium type for a year. Manic-depressive psychosis was diagnosed, in the light of the recurrence of problems of manic attack type (two or three episodes in all).

In 2006, 2007 and 2008, she exhibited paroxymal problems with walking described as balance problems associated with weakness of the lower limbs, which each time lasted less than 24 hours.

The 1^{st} episode in 2006 was accompanied by a fever and the lumbar puncture carried out at the time showed 17 elements (lymphocytes), a CSF protein level of 0.3 g/l.

Between these episodes, she continued to experience discrete balance problems.

At the end of 2006 a painless bilateral decrease in visual acuity occurred, predominantly on the right side, which worsened in stages between 2006 and 2007 (in 2007, she could only count fingers on the right and the left visual acuity was evaluated at 2/10^{th}).

During the summer of 2008, the balance problems became permanent.

The clinical examination in May 2009 showed a static, but also kinetic, discrete right upper limb cerebellar syndrome. The deep tendon reflexes were sharp in the four limbs, but there was no Babinski sign.

The brain MRI showed a leukoencephalopathy made up of periventricular hypersignals, of pyramidal tracks at the level of the cerebral peduncles and of the diencephalon. This leukoencephalopathy also affected the optic radiations and, to a lesser extent, the cerebellar peduncles (FIG. 2A).

The visual evoked potentials carried out in June 2009 showed no cortical response. The visual acuity numbers were 1 on the right, 2/10 on the left. At the time, the patient also complained of frequent headaches (at least one attack per week). Treatment with biotin (3×100 mg/day) was begun in June 2009.

In September 2009, the patient noted an improvement in her visual acuity: she could read telephone numbers, she distinguished faces and could read newspaper headlines. She indicated a considerable decrease in the frequency of the headaches: 1 to 2/month. The balance was better, in particular when turning round. She was able to cook alone, which was not the case previously.

The MRI was unchanged, as was the brain MRI spectro. On the other hand, the visual evoked potentials showed the reappearance of a P100 wave on the left (no response was noted on the right) with a prolonged latency (126.5 ms).

The treatment was continued at the same dose. In January 2010 (after six months of treatment), the evoked potentials showed the beginnings of a P100 wave on the right and also an improvement in the latency of the left P100 wave (which went from 126.5 to 111.8 ms). The brain MRI spectro showed a clear decrease in the choline peak and in the choline/creatine ratio, whereas the leukoencephalopathy remains unchanged (FIG. 2B). The biotin treatment was increased to 600 mg/day. This treatment is still ongoing.

### Example 3

A 40-year-old patient (born in 1970) experienced several transient episodes of double vision for which he had undergone a consultation in 2004, without it being possible to find an etiology.

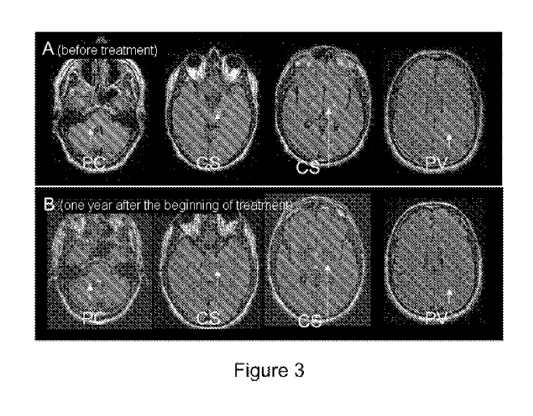

In January 2007, he woke up in the morning with dysarthria having the false appearance of inebriation. This dysarthria worsened slightly between January and February 2007. In March 2007, after hospitalization, the clinical examination simply showed ataxic dysarthria and also a slight static cerebellar syndrome. The clinical examination was otherwise normal. The lumbar puncture showed an inflammatory fluid with 7 elements/mm³ (lymphocytes). The brain MRI showed a leukoencephalopathy affecting the corticospinal tracts at the level of the internal capsules and of the brain stem, the optic radiations and, more weakly, the periventricular white matter (FIG. 3A). Visual acuity was normal.

In March 2007, a biotin treatment (200 mg/day) was begun for a total period of two months. Although no clinical improvement was observed, the control MRI carried out in January 2008 showed a complete disappearance of the leukoencephalopathy (FIG. 3B). The visual evoked potentials showed cortical components of normal latency and morphology on the right, but of reduced amplitude. On the left, the cortical response was dispersed. These results were in favor of a subclinical dysfunction of the left visual pathways.

This patient did not attend follow-up from October 2008 to July 2010 and stopped his treatment during this period of time. His neurological conditioned worsened, in particular his dysarthria and his balance problems, and his worsening resulted in the use of a wheelchair. During this period of time, atrophy of the brain stem and of the cerebellum appeared on the brain MRI. Recommencement of the treatment made it possible to stabilize this patient's condition.

### Example 4

A 49-year-old patient, born in 1961, had a history of bilaterial posterial uveitis complicated by chorioretinitis and bilateral cataracts. No etiology was found despite a very complete work-up. She is overweight and has type 2 diabetes. She has been monitored in psychiatry since 1998 for psychotic disorders which appeared following the birth of her son. Since this time, she has been repeatedly hospitalized for delirious episodes with dream-like hallucinations. An MRI was carried out in the context of the work-up for her disease and shows a leukoencephalopathy affecting the corticospinal tracts, the cerebellar peduncles, the optic radiations and the periventricular white substance. The clinical examination showed very discrete cerebellar ataxia but nothing more.

In September 2008, a biotin treatment (100 mg twice a day) was begun. Three months later (December), the control MRI showed a significant decrease in the choline peak, but with no modification of the leukoencephalopathy. The patient subsequently no longer attended for follow-up.

### Conclusion

The following Table I summarizes the clinical symptoms and radiological and neurophysiological observations in the patients of examples 1 to 4.

TABLE I
characteristics before treatment

| Patient | 1 | 2 | 3 | 4 |
|---|---|---|---|---|
| Clinical characteristics | | | | |
| Sex | F | F | M | F |
| Age at last examination | 53 | 71 | 38 | 47 |
| Age at beginning of problems | 46 | 68 | 34 | 37 |
| Bilateral visual acuity | + | + | − | + |
| decrease | | | | |
| Cerebellar syndrome | + | + | + | + |
| Pyramidal syndrome | + | − | − | − |
| Psychiatric problems | − | + | − | + |
| Headaches | + | + | − | − |
| Radiological, | | | | |
| electrophysiological and | | | | |
| biological characteristics | | | | |
| Periventricular | + | + | − | + |
| leukoencephalopathy | | | | |
| Optical radiation hypersignal | + | + | + | + |
| Cerebellar peduncle hypersignal | + | + | − | + |
| Corticospinal tract hypersignal | + | + | + | + |
| Choline elevation (NMRS) | + | + | ND | + |
| VEP: delay of the 100 waves or | + | + | + | ND |
| lack of response | | | | |
| EMG | NI | NI | NI | NI |
| Elements per mm³ of | 8 | 17 | 7 | 3 |
| cerebrospinal fluid (CSF) | | | | |
| Proteins in the CSF | 0.72 | 0.3 | 0.5 | 0.34 |

ND: not done

Thus, all the patients showed a clinical or paraclinical improvement following the introduction of a treatment with biotin at high doses.

This improvement related to the clinical manifestations (visual acuity and ataxia) in 2 patients/4 (patients 1 and 2), to the MRI abnormalities (decrease in white matter hypersignal) in 2 patients/4 (patients 1 and 3), to the magnetic resonance spectroscopy abnormalities (decrease in the choline peak in the centrum semiovale) in 3 patients/3 (1, 2, 4), and to the visual evoked potentials in 2 patients/2 (patients 1 and 2). It should be noted that the two patients who did not experience a clear clinical improvement with treatment (although the MRI or spectroscopy parameters were improved) showed only very few symptoms before the beginning of treatment.

Not all the patients had the same follow-up time under treatment (between 3 months and 1 year). Nevertheless, it appears that the first signs to be improved are:
1) the decrease in visual acuity in the patients showing a reduction before treatment (patients 1 and 2);
2) the decrease in the choline peak evaluated by NMR spectro and the improvement in the visual evoked potentials, then
3) after at least a year, the decrease in the hypersignal of the white matter in MRI (observed in the two patients followed up with MRI for a year after treatment (patients 1 and 3)).

TABLE II
results after treatment

| | Improve- | Improve- | Improve- | Improve- | |
|---|---|---|---|---|---|
| | ment | ment | ment | ment | Improve- |
| | visual | cerebellar | leucopathy | choline | ment |
| Patient | acuity | syndrome | (MRI) | peak | VEPs |
| 1 | + | + | + | + | + |
| 2 | + | +/− | − | + | + |
| 3 | − | − | + | ND | ND |
| 4 | − | − | − | + | ND |

ND: not done

### REFERENCES

Dabbagh O, Brismar J, Gascon G G, Ozand P T. The clinical spectrum of biotin-treatable encephalopathies in Saudi Arabia. Brain Dev. 1994; 16 Suppl: 72-80.
Debs R, Depienne C, Rastetter A, Bellanger A, Degos B, Galanaud D, Keren B, Lyon-Caen O, Brice A, Sedel F. Biotin-Responsive Basal Ganglia Disease (BBGD) in Europeans with novel SLC19A3 mutations. Arch Neurol. 2010 January; 67(1): 126-30.
Ozand P T, Gascon G G, Al Essa M, Joshi S, Al Jishi E, Bakheet S, Al Watban J, Al-Kawi M Z, Dabbagh O. Biotin-responsive basal ganglia disease: a novel entity. Brain. 1998 July; 121 (Pt 7): 1267-79.
Ramaekers V T, Brab M, Rau G, Heimann G. (1993) Recovery from neurological deficits following biotin treatment in a biotinidase Km variant. Neuropediatrics 24: 98-102.
Sedel F, Tourbah A, Fontaine B, Lubetzki C, Baumann N, Saudubray J M, Lyon-Caen O. Leukoencephalopathies associated with Inborn Errors of Metabolism in adults: a diagnostic approach. J Inherit Metab Dis. 2008 June; 31(3): 295-307.
Subramanian V S, Marchant J S, Said H M. Biotin-responsive basal ganglia disease-linked mutations inhibit thiamine transport via hTHTR2: biotin is not a substrate for hTHTR2. Am J Physiol. 2006; 291(5): 851-859.
Vlasova T I, Stratton S L, Wells A M, Mock N I, Mock D M. Biotin deficiency reduces expression of SLC19A3, a potential biotin transporter, in leukocytes from human blood. J Nutr. 2005; 135(1): 42-47.
Wolf B, Pomponio R J, Norrgard K J, Lott I T, Baumgartner E R, Suormala T, Ramaekers V T, Coskun T, Tokatli A, Ozalp I, Hymes J. J Pediatr. 1998; 132(2): 362-5.
Zempleni J, Wijeratne S S, Hassan Y I. Biotin. Biofactors 2009; 35(1): 36-46.
Zeng W Q, Al-Yamani E, Acierno J S Jr et al. Biotin-responsive basal ganglia disease maps to 2q36.3 and is due to mutations in SCL19A3. Am J Hum Genet. 2005; 77(1): 16-26.

1. A composition for oral administration, containing more than 20 mg of biotin per unit dose.

2. The composition as claimed in claim 1, characterized in that it is in the form of gel capsules, tablets (optionally film-coated), lozenges or pills.

3. The composition as claimed in claim 1, characterized in that it contains biotin and excipients, without any other active ingredient.

4. The composition as claimed in claim 1, characterized in that it contains excipients chosen from talc and microcrystalline cellulose.

5-8. (canceled)

9. A method for the treatment of a visual impairment or of a visual atrophy comprising administering biotin to a patient in need thereof.

10. The method for the treatment of a visual impairment as claimed in claim 9, wherein the amount of biotin administered to the patient is at least equal to 1 mg/kg/day.

11. The method for the treatment of a visual impairment as claimed in claim 9, wherein the visual impairment is related to leukoencephalopathy involving the following regions of the white matter of the brain: periventricular white matter, optic radiations, corticospinal tracts, cerebellar peduncles, as observed by brain MRI, and an elevation of the choline peak in the centrum semiovale, as observed by nuclear magnetic resonance spectroscopy.

12. A method for the treatment of a neurological pathological condition related to an energy metabolism disorder comprising administering biotin to a patient in need thereof.
